(19) United States
(12) Patent Application Publication
(10) Pub. No.: US 2005/0037434 A1
(43) Pub. Date: Feb. 17, 2005

(54) Monoclonal antibody against costimulatory molecule M150 and a process for preparation thereof

(75) Inventors: Durbaka Vijaya Raghava Prasad, Pune (IN); Pradeep Bhaskar Parab, Pune (IN); Gyan Chandra Mishra, Pune (IN)

Correspondence Address: DRINKER BIDDLE & REATH, ONE LOGAN SQUARE, 18TH AND CHERRY STREETS, PHILADELPHIA, PA, 19103-6996, US

(73) Assignee: National Centre for Cell Science, Pune (IN)

(21) Appl. No.: 10/917,938

(22) Filed: Aug. 13, 2004

Related U.S. Application Data
(60) Provisional application No. 60/495,755, filed on Aug. 14, 2003.

(51) Int. Cl. C12Q 1/68; G01N 33/53; C12N 5/06

(52) U.S. Cl. 435/7.1; 530/388.25; 435/336

(57) ABSTRACT

The present invention relates to a monoclonal antibody directed against the 150 kDa protein M150 on activated macrophages.

### FIELD OF INVENTION

The present invention relates to monoclonal antibodies that recognize and specifically bind to M150 kDa protein which is the glycosylated form of lysosome associated membrane protein-1 (LAMP-1) present on activated macrophages. The said antibodies of the invention are useful as immunological and diagnostic reagents. The invention also provides a process for the preparation of a monoclonal antibody directed against M150 kDa protein, which is a glycosylated form of LAMP-1 protein, using a specially designed hybridoma cell line designated as F50.73.66.

### BACKGROUND OF THE INVENTION

The hallmark of the adaptive immune system is its ability to induce as well as regulate its responses to a given infectious agent. Immune responses are of two types: natural immune response and acquired immune response. The natural immune response works through phagocytes such as macrophages and polymorphonuclear leukocytes.

The acquired immune response works by expulsion of infective pathogens by involving T (thymus originated) and B (bone marrow originated) cells which are subsets of lymphocytes. T lymphocytes further differentiate into Cluster of Differentiation (commonly referred as CD in conjugation with a numerical indicator) CD4 and CD8 class. CD4 T cells further differentiate into Th-1 and Th-2 subsets. Th-1 lymphocytes contributing to cellular immunity are characterized by the production of Interleukin (IL) IL-2 and Interferon gamma (INF-γ), whereas Th-2 lymphocytes produce IL-4, IL-5, IL-10 and IL-13, which are known to be involved in humoral immunity. Th-1 and Th-2 associated cytokines tend to be reciprocally regulatory. Activation of CD4+T cells minimally requires two distinct signals to be provided by Antigen Presenting Cells (APC). Although the first signal is provided by the Major Histocompatability (MHC class II) molecules presenting the appropriate antigen derived peptide, the second signal is delivered by a class of accessory molecules that are known as co-stimulatory molecules.

A number of such co-stimulatory molecules have now been identified, along with their counter receptors on T cells. The most prominent among these is CD80, CD86, B7.1 and B7.2, which interact with CD28 receptors on T cells. These molecules are reportedly involved in regulation of immune response. Thus, the immune regulation is far more complex than hitherto suspected and is dependant on an intricate network of interactions between co-stimulatory molecules and appropriate receptors on T-cells.

A novel protein, M150 protein, was isolated by the inventors from the plasma membrane of activated macrophages and was found to deliver co-stimulatory signals to Th cells. It was also found that the M150 protein is expressed only on the surface of activated macrophages. However, the role of the M150 protein in immunomodulation was not known. The inventors have now found that the M150 protein is a specific posttranslational isoform of constitutively produced Lysosome-Associated Membrane Protein-1 (LAMP-1). It is further found that the activity of the said molecule depends upon its unique pattern of glycosylation that is generated only in activated macrophages. (J. Immunology, vol. 169, 1801-1809, 2002). Based on this finding, the invention provides uses of the M150 protein in immunomoregulation, antibodies that recognize this protein and potential application of these antibodies as immunoregulatory and diagnostic reagents in investigation of the Th pathway.

### OBJECTS OF THE INVENTION

The main object of the present invention therefore is to provide monoclonal antibodies that specifically recognize and bind to the M150 kDa protein in its glycosylated form having sequence ID No.1.

Another object is to provide a process for the preparation of said monoclonal antibodies.

Yet another object is to provide hybridomas useful in producing monoclonal antibodies.

### DESCRIPTION OF THE FIGURES

FIG. 1A represents the Western blot analysis of macrophage membrane fractions.

FIG. 1B shows the Western blot analysis of purified LAMP-1-Fc fusion proteins.

FIG. 1C shows deglycosylation of purified LAMP-1-Fc fusion proteins

FIG. 1D shows the lanes 1 and 2, deglycosylated CHO-LAMP-1-Fc and macrophage-LAMP-1Fc products (shown in 1C) probed with anti-Fc Ab.

FIG. 2 is a CD4⁺T cell proliferation assay and cytokine analysis in response to LAMP-P-1Fc.

FIG. 2A shows induction of CD4⁺T cell proliferation induced by macrophage-LAMP-1-Fc.

FIG. 2B shows that anti-M150 blocks the T cell proliferation induced by macrophage LAMP-1-Fc.

FIG. 2C shows induction of Th1 type cytokines by macrophage-LAMP-P-1-Fc.

FIG. 3 shows induction of T-bet, a Th1-specific transcription factor.

FIG. 4 shows portions of the sequence of the M150 protein and sequence identity with portions of LAMP-1. Peptide sequence 1 (SEQ ID NO:2) was derived from the amino-terminal end of M150. Peptide sequences 2 (SEQ ID NO:3) and 3 (SEQ ID NO:4) are internal sequences derived by tryptic digestion of M150.

### DETAILED DESCRIPTION OF THE INVENTION

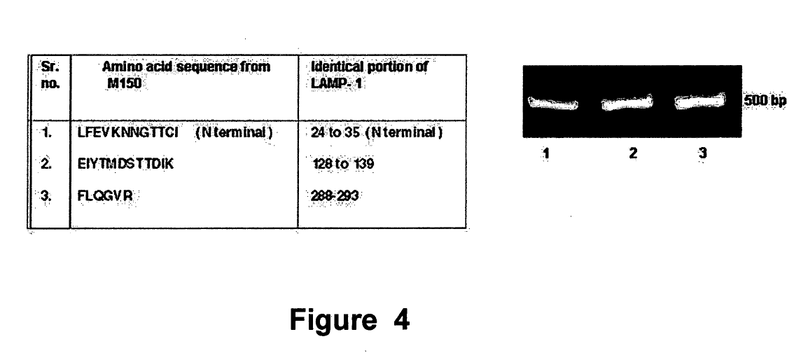

Accordingly, the invention provides monoclonal antibodies that specifically bind to and recognize M150 protein which is a glycosylated form of LAMP-1 protein. The M150 amino acid sequence is depicted in SEQ ID NO:1. Further, an exemplary hybridoma line designated as F50.73.66 that secretes such a monoclonal antibody is also provided. Hybridoma F50.73.66 was deposited at the American Type Culture Collection, Manassas, Va., 20110-2209 USA under accession number PTA-6080 on Jun. 16, 2004. The mouse receptor protein M150 present on the surface of activated macrophages is represented by FIG. 4 and SEQ ID NO:1

Antibodies of the invention (anti-M150) are generated against M150 protein (which is a glycosylated form of LAMP-1). The antibodies are of IgM isotype, with a kappa light chain. The monoclonal antibodies may be produced by injecting a rat with an M150 antigen, collecting the spleen lymphocytes, fusing collected lymphocytes with myeloma cells and culturing them to obtain colonies of hybridoma that produce a virtually endless supply of monoclonal antibodies directed against M150 protein in its glycosylated form.

It has been demonstrated that pathogens such as M. tuberculosis and Leishmania, within hours of infection in macrophages, downregulate co-stimulatory molecules, including M150. The physiological significance of this observation is the inability of parasitized macrophages in providing help to host T-helper cells to generate parasite-specific immune response. Antibody (anti-M150) of the invention has been demonstrated to block the rescuing effect of immunoincapacitation of macrophages infected with M. tuberculosis and Leishmania, and thus provide protection against these infections.

As described above, although a living body activates its acquired immune response system against antigens that are foreign bodies to the living body (self), the body also has immunological tolerance, so as to show no immune response against its own components (autoantigens). If immunological tolerance breaks down for some reason, immune response to autoantigens occurs, and autoantigen-reactive T cells are induced by the same mechanism as mentioned above. The body falls into abnormal state of immunity, and various autoimmune diseases are caused.

In other words, when the immune system of a living body is normal, T cells are in an unresponsive state. They maintain immunological tolerance, even if a few autoantigen-reactive T cells, which react with autoantigen, exist. It has been suggested that in an abnormal state of immunity which involves costimulatory molecules, more autoantigen-reactive T cells are activated due to an abnormal excess of autoantigens, thereby causing autoimmune diseases.

From such a viewpoint, many attempts have been recently made to treat various autoimmune diseases by modulating the transmission of costimulatory signals.

Accordingly, the invention provides a method of treating an autoimmune disease in a experimental subject, the method comprising administering to the experimental subject an effective amount of a pharmaceutical composition comprising (i) a pharmaceutically acceptable carrier and (ii) a monoclonal antibody that blocks the costimulatory molecule and thus block Th1 response induced by autoreactive T cells. The antibody may be generated by the hybridoma line F50.73.66. The autoimmune diseases capable of being treated by the invention are diabetes, arthritis, and so on.

Usually, T cells are in a resting state or a state of unresponsiveness. When they receive a stimulation from APC (antigen presenting cells) or MHC (major histocompatibility complex), they become activated and start producing interleukins. They also start differentiating into antigen specific T cell clones, and proliferate. Similarly, macrophages are inactive and become activated upon receiving stimulation and undergo cell division. When T-cells and macrophages are in an active state, they are called “activated T-cells” and “activated macrophages”.

The invention further provides a fusion protein LAMP-1-Fc comprising a first region having binding specificity for LAMP-1 protein operatively linked to a second region corresponding to an immunoglobulin constant region (Fc).

In one embodiment, the monoclonal antibody of the invention (for M150 kDa protein), may be prepared by:
a. immunizing Lewis rats with intraperotonial injections of M150 antigen by giving at least three booster doses after 21 days of a primary immunization, at an interval of about 2 weeks each, to obtain enrichment of B cells;
b. removing the spleen on the third day after the final booster dose;
c. fusing the spleen cells with SP₂/01- AG14 myeloma cells obtained from American Type Culture Collection USA (No: ATCC CRL 1581), using a fusing agent, to obtain a mixture of hybridomas and unfused cells (mixture);
d. growing the above mixture in Hypoxanthine Aminopterin Thymidine medium (HAT) for at least seven days to obtain colonies of hybridomas;
e. cloning the hybridomas obtained as given in step (d) with conventional methods, such as limiting dilution; and
f. testing the supernatant for the presence of specific antibodies to M150, antibody class IgM, followed by growing the IgM clones in DMEM/FCS (Dulbecco's Minimum Essential Medium with 10% Fetal calf serum ) medium to obtain antibodies.

The fusing agent used is polyethylene glycol having molecular weight 1500-4000. The monoclonal antibody for M150 kDa protein is used for demonstrating in vivo immunoregulatory control in pathogenesis of intracellular pathogens, such as Leishmania, Tuberculosis, Malaria etc. The M150 protein has been identified as a specific post-translational isoform of the constitutively produced lysosome-associated membrane protein 1 (LAMP-1) of SEQ ID NO:1.

Th1 cells help to remove microorganisms such as Mycobacterium leprae, Mycobacterium tuberculosis, and Leishmania, the protozoan that causes leishmaniasis. These organisms live inside lysosomes and phagosomes of macrophages through which they enter. How well the body responds to these organisms depends on lymphokines or cytokines being produced by T-lymphocytes. Cytokines such as interleukin-2 (IL-2) and interferon-gamma (IFN-gamma) produced by Th1 lymphocytes activate macrophages, enabling them to destroy intracellular pathogens.

When Th1 cells produce interferon-gamma, this prompts macrophages to produce toxic substances that destroy microorganisms. On the other hand, when Th2 cells produce IL-4 and IL-10, these cytokines block the microbe killing that is activated by interferon-gamma. Therefore, in order to develop vaccines and other agents to destroy invading pathogens, and determine the efficacy of these products, it is necessary to study Th pathways and immunoregulation in detail.

The antibodies of the present invention are useful diagnostic reagents in this field, and may assist in demonstrating in vivo immunoregulatory control in the pathogenesis of intracellular pathogens such as Leishmania, Tuberculosis, Malaria, etc. The Th1 immune response particularly mediated by macrophages may be selectively blocked using the monoclonal antibodies of the invention, thus providing for delineation of the protective role of other pathways in the control of intracellular pathogens.

The M150 protein has been identified as a specific post-translational glycosylated isoform of the constitutively produced lysosome-associated membrane protein 1 (LAMP-1). The co-stimulatory activity of M150 protein depends upon its unique pattern of glycosylation that is generated only in activated macrophages. This protein also is able to induce secretion of lymphokines that are typical of the Th1 response.

The present invention is illustrated with reference to the following examples which are illustrative only and should not be construed to limit the scope of the present invention in any manner.

### EXAMPLE 1

### Preparation and Purification of M150—the Co-Stimulatory Molecule

Macrophage membrane preparation: Peritoneal exudates cells were harvested from BALB/C mice injected 4 days previously with 2 ml. of 3% thioglycolate. The peritoneal exudates cells were washed with cold HBSS and macrophages were obtained by adhering for 1 hour at 37° C. on plastic petry dishes. The purity of macrophage was ≧98% as analysed by their reactivity with anti mac-1 antibody. The macrophages were washed thrice and the pellet was frozen overnight at −70° C. The cells were thawed and homogenized in the presence of 20 mM Tris-HCL(Ph 7.4) and 1 mM EDTA, along with a protease inhibitor mixture (10 μg/ml leupeptin, 10 μg/ml aprotinin, 10 μg/ml pepstatin, 10 μg/ml of antipain, 10 mM iodoacetamide, 10 μg/ml chymostatin and 1 mM PMSF). The nuclear fraction was removed by centrifuging at 700 g for 10 minutes at 4° C. The supernatant was subjected to centrifugation at 110,000×g for 2 hours at 4° C. The pellet was solubilised overnight 20 mM Tris HCl, pH 7.5, containing 1% Triton x-100, 20% glycerol and protease inhibitor mixture, and re-centrifuged at 100,000×g for 1 hour at 4° C. to remove insoluble debris. The membrane proteins in the supernatant were separated on 10% SDS-PAGE. M150 protein was eluted from the gel by crushing the gel pieces containing the protein band followed by overnight incubation with 100 mM ammonium bicarbonate, 50 mM Tris-HCl, 0.1 mM EDTA and 150 mM NaCl (pH8.0). SDS from the protein solution was removed by passage through an Extracti-D gel column. The protein content was estimated using BCA Kit. The protein was further purified by FPLC on a Mono-Q column, and the purity was ascertained by 2D-Gel Electrophoresis.

### EXAMPLE 2

### Development of Monoclonal Antibody against M150

Lewis rat 8-10 weeks old, immunized by intraperitoneal injections of thioglycolate, elicited macrophages(10⁷ cells per mice) from BALB/c mice. Three booster doses were given after 21 days of primary immunization at an interval of 2 weeks each. The spleen was removed on the third day after the final booster dose and the cells were fused to SP2/01-AG14 mouse myeloma cells using Polyethylene Glycol 1500 MW. Hybrids were selected in Hypoxanthine/Aminopterin/Thymidine medium. The supernatants were screened for M150 reactivity both by ELISA and Western Blot analysis. Positive clones were subcloned by limiting dilution. The isotype of antibodies secreted by individual clones was determined using the monoclonal based Rat Ig isotyping Kit. Cells from positive clones were then expanded in DMEM 10% FCS and then cryopreserved in freezing medium (90% FCS, 10% DMSO mixture) and stored in liquid nitrogen.

### EXAMPLE 3

### Differential Glycosylation Gives Antigenic Distinctness to LAMP-1

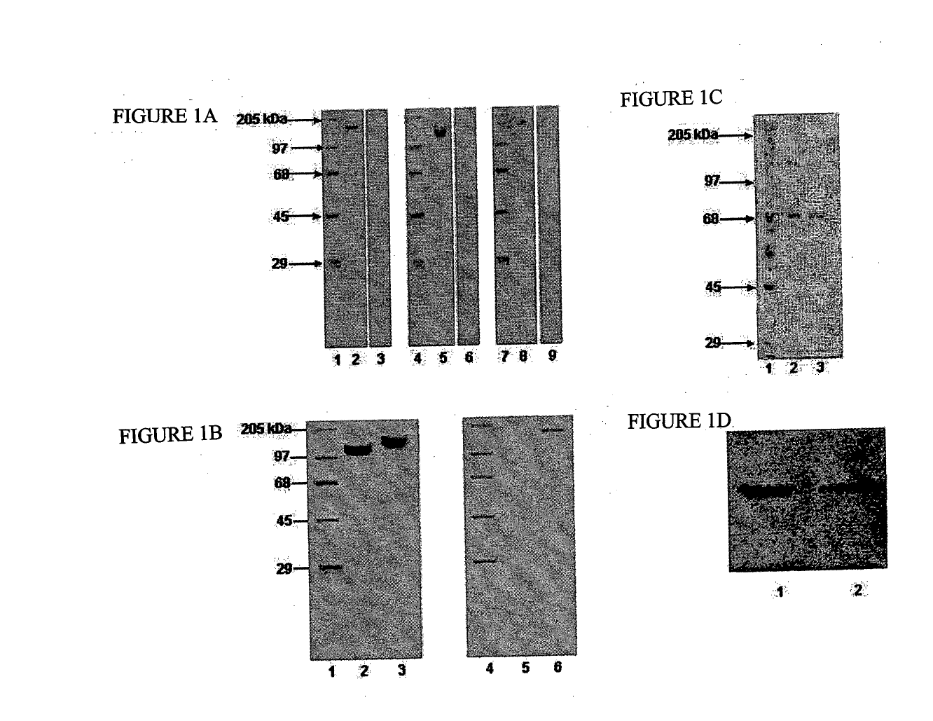

The existence of antigenic variance between M150 and LAMP-1 was also confirmed by western blot analysis of the membrane fraction of activated macrophages, using either anti-M150 or anti-LAMP-1 antibodies as probes. Probing with either polyclonal or monoclonal anti-M150 antibody (G1) detected a band that centered around a molecular weight of 150 kDa. As opposed to this, anti-LAMP-1 antibody identified a lower band ranging from 105 to 120 kDa (FIG. 1A). Previous reports have demonstrated that the size heterogeneity of LAMP-1 is due to heterogeneous glycosylation (Chen. J. W., Y. Cha, K. U. Yuksel, R. W. Gracy, and J. T. August, 1988. Isolation and sequencing of a cDNA clone lysosomal membrane glycoprotein mouse LAMP-1: sequence similarity to proteins being onco-differentiation antigens. J. Biol. Chem. 263:8754). These studies also revealed that the size distribution of LAMP-1 differs significantly, depending upon the cell type examined. In other words, the glycosylation pattern of LAMP-1 appears to vary depending upon the cell type in which it is expressed (Chen. J. W., W. Pan, M. P. D'Souza, and J. T. August. 1985. Lysosome-associated membrane proteins: characterization of LAMP-1 of macrophage P388 and mouse embryo 3T3 cultured cells. Arch. Biochem. Bioplys. 239:574).

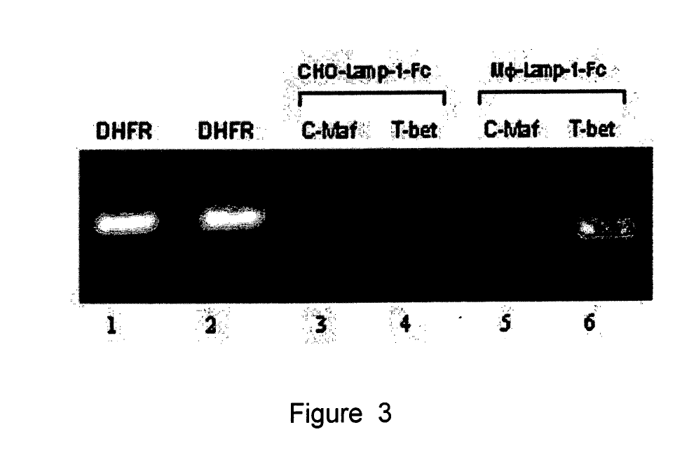

To further confirm the possible cell type specific differences in glycosylation of LAMP-1, we expressed a truncated version of LAMP-1 protein as LAMP-1-Fc chimera, either in CHO cells, or in the mouse macrophage cell line, P388D1. The transfected fusion proteins were then purified and subjected to a western blot analysis. As shown in FIG. 1B, non-identical forms of the chimeric protein were produced in CHO and P388D1 cells, at least as detected by anti-LAMP-1 and anti-M150 monoclonal antibodies. The product of CHO cells displayed a molecular mass between 105 to 120 kDa; in contrast, the product from P388D1 cells was significantly of higher molecular weight, ranging from 130 to 150 kDa. Interestingly, anti-M150 mAb did not show reactivity with LAMP-1 protein expressed in CHO cells while recognizing the LAMP-1 expressed in macrophages (compare lanes 5 and 6 FIG. 1B). In contrast, anti-LAMP-1 recognized discrete number of lower molecular weight proteins from LAMP-1 transfected CHO and P388D1 cells as shown in FIG. 3B lanes 2 and 3, respectively. A comparison of the protein bands (FIG. 1B lane 3 and 6) recognized by anti-LAMP-1 and anti-M150 antibodies suggest that M150 is a specific form, which constitutes only a small fraction of the total LAMP-1 produced in macrophages.

In order to compare the protein core of these two molecular species, the expressed products from both CHO and P388D1 cells were first deglycosylated using endoglycosidase H. Surprisingly, this procedure completely eliminated the reactivity of the anti-LAMP-1 and anti-M150 antibodies. Thus, the resultant deglycosylated proteins could be detected only with anti-Fc antibody.

These results, therefore, collectively suggest that both antibody preparations are directed against the glycosylated portion of the molecules. Thus, from these results, it can also be inferred that the expression of LAMP-1-Fc chimera in CHO and P388D1 cells results from qualitatively distinct glycosylation patterns. Furthermore, it is this variation in glycosylation that accounts for the observed difference in antigenicity, at least with respect to anti-M150 antibody. In other words, the anti-M150 mAb appears to be directed against a LAMP-1 subset that is specifically produced only in macrophages.

### EXAMPLE 4

### Costimulatory Activity of M150 in Macrophages

The following test was carried out, to examine whether the altered glycosylation pattern of the LAMP-1-Fc chimera also confers altered function for the protein molecule when expressed in P388D1.

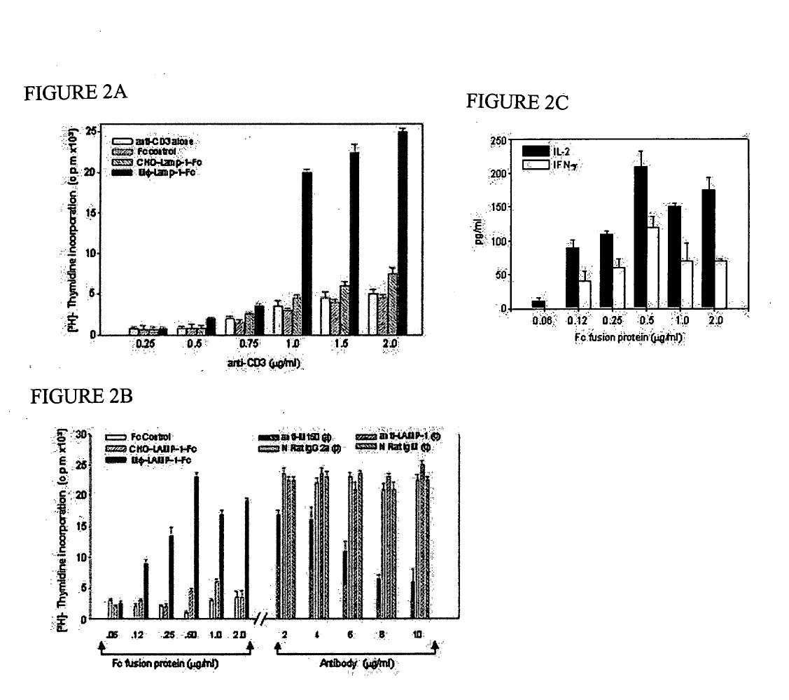

Purified CD4⁺T cells were stimulated with varying concentrations of anti-CD3 antibody in the presence of a fixed concentration of either CHO-LAMP-1-Fc or macrophage-LAMP-1-Fc proteins. A proliferative response was observed only in cultures that included macrophage-LAMP-1-Fc, but not in those where CHO-LAMP-1-Fc was added (FIG. 2A).

It is possible that the difference in activity observed for macrophage-LAMP-1-Fc and CHO-LAMP-1-Fc in FIG. 2A simply reflects differences in dose requirements for the two chimeric products. To verify this, T cells were stimulated with a constant dose of 1 μg/ml of anti-CD3 antibody in the presence of increasing concentrations of either macrophage-LAMP-1-Fc or CHO-LAMP-1-Fc. For these experiments, a dose-dependent proliferation of CD4⁺ T cells was obtained with macrophage-LAMP-1-Fc (FIG. 2B) . In contrast to this, CHO-LAMP-1-Fc was unable to induce T cell proliferation at any of its tested concentrations (FIG. 2B). Further, the specificity of the macrophage-LAMP-1-Fc dependent T cell response is also evident from the fact that this effect could be inhibited by addition of anti-M150 antibody, but not by anti-LAMP-1 antibody (FIG. 2B).

It has been previously demonstrated that M150 serves as a costimulatory molecule that specifically elicits cytokines typical of the Th1 subset of CD4⁺ T cells. As shown in FIG. 2C, the macrophage-LAMP-1-Fc product also reproduced this activity, when used in conjunction with anti-CD3 for stimulation of CD4⁺ T cells. Significant levels of both IL-2 and IFN-γ were detected in the supernatants from these cultures (FIG. 2C). On the other hand, stimulation in presence of CHO-LAMP-1-Fc failed to induce detectable levels of Th1 representative cytokines (FIG. 2C). It is pertinent to mention that no detectable levels of Th2 representative cytokines were obtained from the culture supernatants of Th cells activated either by CHO-LAMP-1-Fc or macrophage LAMP-1-Fc. Collectively, these results categorically identify that LAMP-1 can indeed display Th1-specific costimulatory activity, but this activity is contingent upon its expression in activated macrophages.

### EXAMPLE 5

### Macrophage-LAMP-1-Fc Induces Expression of T-bet in Naive CD4⁺ T Cells

It is now becoming evident that differentiation of CD4⁺ T cells into either the Th1 or Th2 commitment pathways is regulated by the activation of independent transcription factors (Murphy, K. M., W. Ouyang, J. D. Farrar, J. Yang, S. Ranganath, H. Asnagli, M. Afkarian, and T. L. Murphy. 2000, Signaling and transcription in T helper development. Annu. Rev. Immunol. 18:451). Subset-specific transcription factors have been identified and their role in distinct T cell differentiation is being actively explored. For example, STAT-6 has been shown to synergize with an antigenic stimulus leading to the up regulation of GATA3, a potent inducer of Th2 differentiation (Ouyang, W., M. Lohning, Z. Gao, M. Assenmacher, S. Ranganath, A. Radbruch, and K. M. Murphy. 2000. Stat6-independent GATA-3 autoactivation directs IL-4-independent Th2 development and commitment. Immunity 12:27). The transcription factor c-Maf, which negatively regulates Th1 differentiation, has now been shown to be responsible for the tissue specific expression of IL-4 (Ho, I. C., M. R. Hodge, J. W. Rooney, and L. H. Glimcher. 1996. The proto-oncogene c-Maf is responsible for tissue-specific expression of interleukin-4. Cell 85:973). Recently, T-bet was identified as a transcription factor specific to Th1 cells, and demonstrated to act by controlling the expression of IFN-γ. Given the Th1 specific costimulatory activity observed for M150 in our earlier study (Agrewala, J. N. D. S. Vinay, A. Joshi, and G. C. Mishra, 1994. A 150-kDa molecule of macrophage membrane stimulates interleukin-2 and interferon-γ production and proliferation of ovalbumin-specific CD⁴T cells. Eur. J. Immunol. 24:2092), as well as in the present report (FIG. 2C), we further examined whether it could be implicated in selectively driving differentiation of CD4⁺ T cells.

CD4⁺T cells were stimulated with anti-CD3 in presence of either macrophage-LAMP-1-Fc or CHO-LAMP-1-Fc from which the total RNA was isolated. This was then analyzed for the presence of specific mRNA for c-Maf and T-bet by RT-PCR. Consistent with the absence of any activity of CHO-LAMP-1-Fc, no expression of either c-Maf or T-bet could be detected in cells stimulated with this protein. In contrast, costimulation with macrophage-LAMP-1-Fc yielded a significant induction of T-bet mRNA, with no concomitant effect on c-Maf expression (FIG. 3). This selective effect of macrophage-LAMP-1-Fc on T-bet expression may therefore implicate M150, a modified glycosylated form of LAMP-1, as a costimulatory molecule that specifically drives the naive CD4⁺T cells towards Th1 differentiation.

### EXAMPLE 6

### Fusion Protein LAMP-1-Fc

The vector pCEP4 having an Fc tag of human IgG1 was a gift from T W Mak (Amgen Inc. Calfornia). Truncated LAMP-1 gene devoid of the C-terminal transmembrane domain was cloned under the CMV promoter as a fusion protein with the Fc fragment. Truncated LAMP-1 gene was amplified from the cDNA of LAMP-1 (gift from Dr. J. Thomas August). The LAMP-1 gene was fused inframe to the amino terminus region of the Fc tag.

The highly purified LAMP-1-Fc plasmid was then transfected into P388D1, a macrophage cell line (DSMZ ACC288, Germany) using Fusin-6 transfection reagent(Roche Biochemicals). The soluble secretory protein was affinity-purified using protein A-agarose column chromatography. This recombinant protein has given biological activity comparable to native macrophage protein.

1. A monoclonal antibody useful as an immunological and diagnostic reagent, and capable of specifically binding to M150 protein, which M150 protein is a glycosylated form of LAMP-1 located on the surface of activated macrophages, said M150 protein being of SEQ ID NO:1 and bearing homology to human LAMP-1 protein.

2. The antibody of claim 1 that binds to the surface molecule M150 of activated macrophages.

3. The antibody of claim 1 being of the IgM isotype with Kappa-light chain.

4. The hybridoma cell line ATCC accession No. PTA-6080, capable of producing the antibody of claim 1.

5. The antibody secreted by the hybridoma cell line of claim 4.

6. A process for producing the antibody of claim 5, comprising culturing cells of the hybridoma cell line ATCC accession No. PTA-6080, and obtaining the secreted antibody.

7. A process for the preparation of monoclonal antibody against M150 kDa protein, which antibody is the antibody produced by the hybridoma of ATCC accession No. PTA 6080, the process comprising:
a. immunizing Lewis rats with intra perotonial injections of M150 antigen by giving at least three booster doses after 21 days of primary immunization at an interval of about 2 weeks each to obtain enrichment of B cells;
b. removing the spleen of the rats on the third day after the final booster dose;
c. fusing the spleen cells with the SP₂/01-AG14 myeloma cells of ATCC CRL 1581, using a fusion agent to obtain a mixture of hybridomas and unfused cells;
d. growing the mixture of hybridomas and unfused cells in Hypoxanthine Aminopterin Thymidine medium for at least seven days to obtain colonies of hybridomas;
e. cloning the hybridomas obtained in step; and
f. testing the supernatant for the presence of specific IgM class antibodies against M150, followed by growing IgM-producing clones in Dulbecco's Minimum Essential Medium with 10% Fetal calf serum to obtain antibodies.

8. The process as claimed in claim 7, wherein the fusion agent is polyethylene glycol having a molecular weight of 1500 to 4000.

9. A process for as claimed in claim 2 wherein the monoclonal antibody for M150 kDa protein is used for demonstrating in vivo immunoregulatory control in pathogenesis of intracellular pathogens such as Leishmania, Tuberculosis, Malaria.

10. A process as claimed in claim 1 wherein the Th1 immune response particularly mediated by macrophages can be selectively blocked using F50.73.66 antibody thus providing for delineation of protective role of other pathways in control of intracellular pathogens.

11. A process as claimed in claim 1 wherein the M150 has been identified as a specific post translational isoform of constitutively produced lysosome associated membrane protein 1(LAMP-1).

12. A method for selectively blocking Th1 immune response mediated by macrophages by using F50.73.66 antibody thus providing for delineation of protective role in control of autoimmune diabetes.

13. A method of treating an autoimmune disease in a subject, the method comprising administering to the subject an effective amount of a pharmaceutical composition comprising (i)a pharmaceutically acceptable carrier and (ii) a monoclonal antibody that blocks the costimulatory molecule M150, and thus blocks the Th1 response induced by autoreactive T cells.

14. The method as claimed in claim 13, wherein the antibody is a monoclonal antibody as claimed in claim 1.

15. The method as claimed in claim 13, wherein the autoimmune disease is type 1 diabetes.

16. The method as claimed in claim 13, wherein the antibody binds to the extracellular region of the polypeptide M150, which polypeptide has the amino acid sequence SEQ ID NO:1.

17. The method as claimed in claim 13, wherein the monoclonal antibody comprises the monoclonal antibody secreted by the hybridoma cell line ATCC PTA-6080.


SEQUENCE LISTING (4 sequences)

SEQ ID NO: 1 - protein, 405 aa, Mus musculus
MRPPRAAAVLLLLLAGLAHGASALFEVKNNGTTCIMASFSASFLTTYETANGSQIVNISLPASAEVLKNGSSCGKENVSDPSLTITFGRGYLLTLNFTKNTTRYSVQHMYFTYNLSDTEHFPNAISKEIYTMDSTTDIKADINKAYRCVSDIRVYMKNVTVVLRDATIQAYLSSGNFSKEETHCTQDGPSPTTGPPSPSPPLVPTNPTVSKYNVTGNNGTCLLASMALQLNITYLKKDNKTVTRAFNISPNDTSSGSCGINLVTLKVENKNRALELQFGMNASSSLFFLQGVRLNMTLPDALVPTFSISNHSLKALQATVGNSYKCNTEEHIFVSKMLSLNVFSVQVQAFKVDSDRFGSVEECVQDGNNMLIPIAVGGALAGLILIVLIAYLIGRKRSHAGYQTI

SEQ ID NO: 2 - protein, 12 aa, Artificial Sequence
  M150 N terminal sequence
LFEVKNNGTTCI

SEQ ID NO: 3 - protein, 12 aa, Artificial Sequence
  M150 internal sequence
EIYTMDSTTDIK

SEQ ID NO: 4 - protein, 6 aa, Artificial Sequence
  M150 internal sequence
FLQGVR